(19) United States
(12) Patent Application Publication
(10) Pub. No.: US 2015/0105708 A1
(43) Pub. Date: Apr. 16, 2015

(54) STRAP STICKER FOR BODY ADHESION

(71) Applicant: NATIONAL TAIWAN UNIVERSITY, TAIPEI (TW)

(72) Inventors: Tai-Horng YOUNG, Taipei (TW); Hsu-Hsien CHANG, Taipei (TW); Ya-Shuan CHOU, Taipei (TW)

(73) Assignee: NATIONAL TAIWAN UNIVERSITY, TAIPEI (TW)

(21) Appl. No.: 14/149,581

(22) Filed: Jan. 7, 2014

(30) Foreign Application Priority Data
Oct. 16, 2013 (TW) 102137241

(51) Int. Cl. A61F 5/01

(52) U.S. Cl. CPC A61F 5/0102
USPC 602/5

(57) ABSTRACT

A strap sticker for body adhesion is provided with an outer layer and an inner layer of different elastic coefficients, and an observation well running through both the inner layer and the outer layer, a well cross section profile of the observation well and a level for inner layer material coming out from the observation well changing with a force bearing status of a body of the strap sticker. Thereby, naked eyes may be used to identify directly a force bearing extension level of the strap sticker to meet the purpose of home use.

### CROSS-REFERENCE TO RELATED APPLICATIONS

This application claims the priority of Republic of China Patent Application No. 102137241 filed on Oct. 16, 2013, in the Intellectual Property Office Ministry of Economic Affairs Republic of China, the disclosure of which is incorporated herein by reference.

### BACKGROUND OF THE INVENTION

1. Field of the Invention

The invention relates to a strap sticker, more specifically, a strap sticker for body adhesion.

2. Descriptions of the Related Art

In daily life, people may feel fatigue and sore with respect to joint muscle due to over work. Permanent hurt is possible in the long term if the fatigue and sore feelings with respect to joint muscle are not cared well. Therefore, someone develops an elastic strap sticker for adhesion to skin on join portion of body, such that the physical property of anti-extension for the strap sticker in longitudinal or transversal direction is utilized to provide support for joint muscle, and thereby, release fatigue and sore joint muscle.

Such elastic strap sticker cannot only be applied to support joint muscle, but also be utilized to recover fascia mechanism and recover joint mechanism, correct tendon, protect joint ligament or protect muscle tissue function, release the symptom of muscle stiffness or sore and inflammation such that affected parts are treated. The application range is wide.

In using existent strap stickers, a certain degree of stretch and extension is necessary to provide sufficient elastic restoring force once they are adhered to human skin, and thereby, to provide support for muscle under skin. Thus, the use strap stickers usually have to be performed by professional medical personnel such that expected medical or rehabilitation effect of strap sticker will not be impacted due to too much or too less force bearing extension level of strap sticker, which cause strap sticker to be unable to be used at home or have too high a cost, which results in poor popularity.

Therefore, it is a problem concerned continuously and necessary to be figured out urgently by those skilled in the art and perspective vendors about how to provide a strap sticker with force bearing extension level identifiable for body adhesion for people to use without the assistance from medical personnel.

### SUMMARY OF THE INVENTION

In view of various problems of above prior art, a purpose of the invention is to provide a strap sticker for body adhesion, in that force bearing extension level of the strap sticker is identified without assistance of medical personnel for elastic force of the strap sticker to provide sufficient support for joint muscle, such that the effect of home medication is achieved.

To achieve above and other purposes, the invention provides a strap sticker for body adhesion. The strap sticker includes an elastic body and a glue film. The elastic body has an outer layer and an inner layer with different elastic coefficients, and an observation well penetrating said outer layer and said inner layer. The glue film lays on one surface of said elastic body. The strap sticker further includes a comparative object marked with comparative patterns, the comparative object being used to provide comparison with shape of opening of an observation well after an elastic body bears force to determine force bearing extension level of the elastic body.

A strap sticker for body adhesion according to the invention further includes an extension level identification tag, which is extended from said inner layer to be exposed to said observation well, well diameter of said observation well in said inner layer being larger than that in said outer layer to form an indent space inside said observation well, such that said extension level identification tag being indented in said indent space to reduce level of exposure to said observation well as said elastic body does not bear force to extend. Similarly, said extension level identification tag being extended outside said indent space and exposed to said observation well as said elastic body bearing force to extend.

Furthermore, the strap sticker for body adhesion according the invention is provided with a force showing bar. The force showing bar is extended on opposed walls on two sides and has a structurally weak point with respect to the reaction to the extension level of the elastic body.

To sum up, the strap sticker provided in the invention may be attached to body skin to provide support for muscle under skin by a pre-stress reserved due to stretch of the strap sticker itself. Compared to prior art, the force bearing extension level of the inventive strap sticker may be identified accurately by observing change for the shape of the opening in the observation well. Besides, a force showing bar or extension level identification tag may be extended from wall of the observation well such that the identification of the force bearing extension level of the strap sticker is more direct by the exposure of the force showing bar or extension level identification tag from the observation well.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a schematic view showing a roll of a strap sticker according to the invention.

FIG. 2 shows a comparative object marked with comparative patterns.

FIG. 3A is a schematic view showing one implementation according to the invention.

FIG. 3B is a schematic view showing a change for shape of a force showing pattern as two sides of the strap sticker as shown in FIG. 3A are pulled.

FIG. 4A is a sectional view of the strap sticker cut along AA line in FIG. 1.

FIG. 4B is a schematic view showing partial material in inner layer of the strap sticker in FIG. 4A to be exposed to the observation well after the strap sticker bears force.

FIG. 5 is a view showing status for use of the strap sticker after extension due to force bearing according to the invention.

FIG. 6A is a schematic view showing an example in that the strap sticker is formed with an extension level identification tag.

FIG. 6B is a top view showing the strap stick in FIG. 6A.

FIG. 7A is a schematic view showing the extension level identification tag exposed to the observation well after the strap sticker as shown in FIG. 6A bears force to extend.

FIG. 7B is a top view showing the strap sticker as shown in FIG. 7A.

FIG. 8A is a schematic view showing an example of the strap sticker provided with a force showing bar according to the invention.

FIG. 8B is a top view of the broken force showing bar after the strap sticker as shown in FIG. 8A bears force.

FIG. 9A is a schematic view showing an example of the strap sticker provided with a force showing bar according to the invention.

FIG. 9B is a top view showing the strap sticker as shown in FIG. 9A with broken force showing bar.

FIG. 10 shows the strap sticker in that a glue film is disposed on a local surface area of an elastic body in a wave shape.

### DETAILED DESCRIPTION OF THE PREFERRED EMBODIMENT

Embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions of elements may be exaggerated for clarity, and the same reference numerals will be used throughout to designate the same or like components.

The invention provides a strap sticker for body adhesion. The composition of the strap sticker is designed optimally for a force bearing extension level of the strap sticker to be identified by naked eyes directly, such that the strap sticker may be utilized without assistance of medical personnel, and thereby, the vision of home use is further achieved.

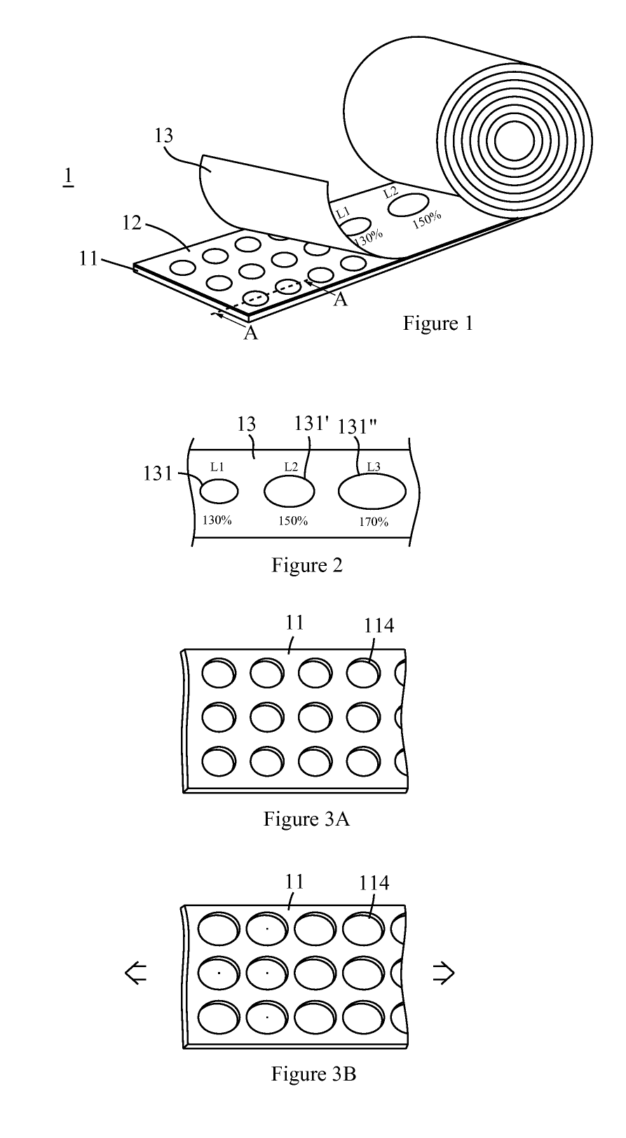

Refer to FIGS. 1, 2, 3A and 3B, which are schematic views showing the strap sticker for body adhesion according to the invention. As shown in the figures, the strap sticker 1 of the invention includes an elastic body 11, a glue film 12 and a comparative object 13. The elastic body 11 is provided with a force showing pattern 114 formed by a through hole of the elastic body 11 or pigment marked on the elastic body 11. The force showing pattern 114 may be of circle, ellipse, polygon or other combined shape, or other pattern shapes with special visual effect.

The glue film 12 is a surface laid on the elastic body 11 opposed to the force showing pattern 114 for providing viscosity to be glued on skin of user. The comparative object 13 shown in FIG. 2 is marked with comparative patterns 131, 131′, 131″, which are used to provide shape comparison of the force showing pattern 114 after the elastic body 11 bears force for force bearing extension level of the elastic body 11 to be determined. The comparative object 13 may be a release paper covering above the glue film 12. Even more, the comparative object 13 may also be, for example, outer packing box, packing paper, user manual, medicine sheet or any paper marked with pattern. The release paper may provide protection of glue film for the unused elastic body 11. As shown in FIG. 2, which shows a comparative object marked with comparative patterns. In FIG. 2, the multiple comparative patterns 131, 131′, 131″ marked on the comparative object 13 correspond to shapes of the observation well 131 as the elastic body 13 is at L1, L2, L3 extension levels, respectively. Wherein, symbols L1, L2, L3 represents that the elastic body 13 has extension levels with 130%, 150%, 170% extension rates, respectively.

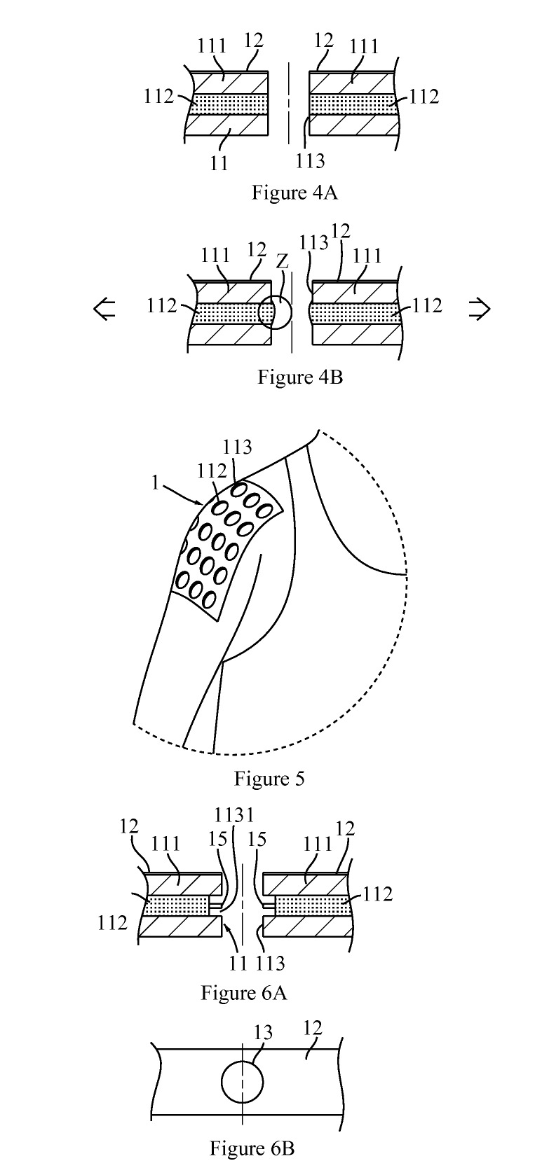

Further, refer to FIGS. 4A, 4B and 5, which are schematic views showing one implementation according to the invention. As shown in FIG. 4A, the strap sticker of the invention includes an elastic body 11 and a glue film 12. The elastic body 11 may be a multi-layer structure with different elastic coefficients. In the implementation, the elastic body 11 has an outer layer 111 and an inner layer 112, and an observation well 113 penetrating through the outer layer 111 and the inner layer 112. Preferably, the inner layer 112 is mixed with a pigment different from color on the outer layer 111, and the elastic coefficient of the inner layer 112 is larger than that of the outer layer 111 such that a material with color different from the outer layer 111 is exposed in the observation well 113 to construct a force showing pattern in the observation well 113 as the inner layer 112 is extended due to force bearing of the strap sticker 1. It should be noted that the more the material of the inner layer 112 exposed in the observation well 113 is, the larger the force bearing at the two ends of the strap sticker 1 is, that is, the extension level of the strap sticker is larger. As such, the force bearing extension level of the strap sticker 1 is identifiable with the exposure status of the inner layer material in the observation well 113. The area indicated by symbol Z shown in FIG. 4B is the material of the inner layer 112 exposed in the observation well 113.

The elastic body 11 may be made of natural fiber material, synthetic fiber material or the arbitrary combination thereof. Preferably, the elastic body 11 may be made of PU (Polyurethane), PE (Polyethylene), PP (Polypropylene), PVC (polyvinyl chloride), EVA (Ethylene Vinyl Acetate copolymer), EAV (Ethylene Vinyl Acetate copolymer), PVA (poly vinyl alcohol), Nylon (Polyamide), TPU (Thermoplastic Polyurethane), PET (Poly(ethylene terephthalate)) or the arbitrary combination thereof. The elastic body 11 includes further a antibacterial material for bacterial removal or bacterial growth prevention; the antibacterial material includes, but not limited to, nanometer silver grain, quaternary salt, chitosan or the combination thereof.

The opening shape of the observation well 113 may be circle, ellipse, polygon, irregular shape or other shape with special visual effect. As the strap sticker 1 bears force to extend, the opening of the observation well 113 will change corresponding to the force bearing level of the strap sticker 1. Thus, the force bearing extension level of the strap sticker 1 may be determined with the opening shape of the observation well 113.

The elastic body 11 shown in FIG. 4A is a three-layer structure, but the invention is not limited thereto, and the layer number of the elastic body 11 may also be changed properly according to the requirement of rehabilitation treatment. Refer to FIG. 5, which is a schematic view showing the usage status of the strap sticker attached to body after being extended to a certain level due to force bearing. The wall of the observation well 113 as shown in FIG. 5 is an inner layer material exposed to outside with circular extension to construct a force showing pattern.

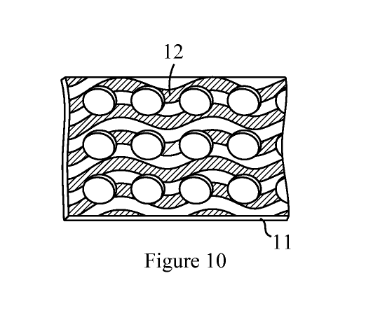

The glue film 12 is laid on the surface of the outer layer 111 of the elastic body 11 for adhesion to skin of user. The glue film 12 has to be a material that does not induce inappropriate situation such as allergic reaction to human skin, including, but not limited to, silicone, acrylic adhesive or the combination thereof. The glue film 12 may be laid over a local surface area of the elastic body 11 in wave form (as shown in FIG. 10) or concentric form such that buckling is generated on skin as the glue film 12 is attached to the skin, and thereby, blood circulation is improved, metabolism is improved, muscle soft tissue is supported or stretched effectively to achieve the function with respect to protection of muscle soft tissue.

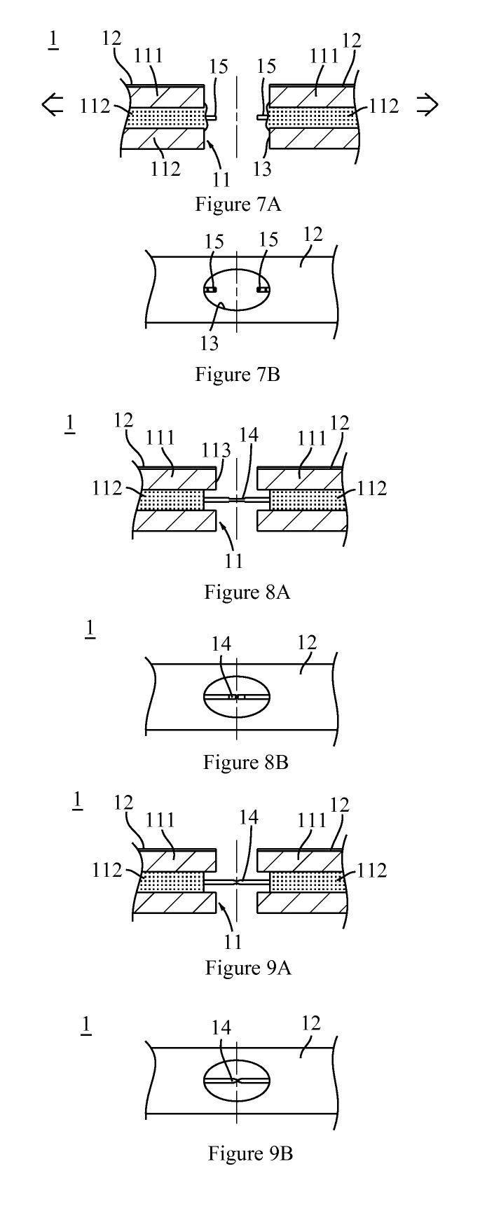

Refer to FIGS. 6A, 6B, 7A, 7B, which are schematic views showing an implementation of the strap sticker according to the invention. FIGS. 6A and 6B show the status as the strap sticker does not bear force to extend. FIGS. 7A and 7B show the status as the strap sticker bears force to extend and exposes the extension level identification tag. As shown in the figures mentioned above, the strap sticker 1 of the implementation is provided with an extension level identification tag 15, which is extended from the inner layer 112 and may be exposed to the observation well 1 by the extended and deformed inner layer 112 as the strap sticker 1 bears force. The more the portion of the extension level identification tag 15 exposed to the observation well 1 is, the larger the force bearing extension level of the strap sticker 1 is. Preferably, the portion or the surface of the extension level identification tag 15 exposed to the observation well 113 may be marked with text or pattern for identifying the current force bearing extension level of the strap sticker 1.

In FIG. 6A, the well diameter of the observation well 113 in the inner layer 112 is larger than that in the outer layer 111 such that an indent space 1131 is formed inside the observation 113. As the elastic body 11 does not bear force for extension, the extension level identification tag 1111 may be indented in the indent space 1131 to reduce the level with respect to exposure to the observation well 113, and expand scenic difference in the observation well 113 before and after the strap sticker 1 bears force to extend, such that the current force bearing extension level of the strap sticker 1 may be identified more effectively.

Also, refer to FIGS. 8A, 8B, 9A and 9B, which are schematic views showing another implementation of the strap sticker according to the invention. In the implementation, the strap sticker 1 is provided with a force showing bar 14 for responding to the extension level of the elastic body 11. As shown in FIGS. 8A, 8B, the strap sticker 1 in the implementation is provided with a force showing bar 14 extended on opposed walls on two sides to separate the opening of the observation well 113 into multiple areas. The force showing bar 14 has a structurally weak point with respect to the reaction to the extension level of the elastic body 11. The structurally weak point indicates the part with smaller thickness in the force showing bar 14 (as shown in FIG. 8A), or the part with smaller cross sectional area (as shown in FIG. 9A), or the part with smaller strength (not shown). As the strap sticker 1 is extended due to force bearing on two ends, forces are conducted to the force showing bar 14 simultaneously until the force showing bar 14 is broken on the structurally weak point, as shown in FIGS. 8B, 9B. As such, whether the extension of the strap sticker 1 has achieved a predetermined extension level is identifiable with the observation about whether the force showing bar 14 is broken.

In conclusion, there are the following features of the strap sticker for body adhesion according to the invention:

1. One or more observation wells are provided in the strap sticker such that the force bearing extension status of the strap sticker is identifiable visually by the change with respect to the shape of the opening in the observation well to meet the goal of using the strap sticker for home use.

2. A force showing pattern with shape changeable according to the force bearing extension of the elastic body is provided on the outer surface of the strap sticker such that the force bearing extension level of the strap sticker may be determined effectively by the force showing pattern along with the comparative pattern marked by the comparative object.

3. The force showing bar or the extension level identification tag are extended in the wall of the observation well of the strap sticker such that the identification for the force bearing extension status of the strap sticker is more direct by the exposure of the force bearing bar or the extension level identification tag in the observation well.

What is claimed is:

1. A strap sticker for body adhesion, including:
an elastic body, having an outer layer and an inner layer with different elastic coefficients, and an observation well penetrating said outer layer and said inner layer; and
a glue film, laying on one surface of said elastic body.

2. The strap sticker for body adhesion as claim 1, wherein, the elastic coefficient of said inner layer is larger than that of the outer layer, and the layer color of said outer layer is different from the layer color of said inner layer.

3. The strap sticker for body adhesion as claim 1, wherein, cross sectional shape of opening of said observation well forms a force showing pattern after said elastic body bears force.

4. The strap sticker as claim 3, further including a comparative object, which is marked with a comparative pattern for providing comparison with said force showing pattern to determine force bearing extension level of said elastic body.

5. The strap sticker for body adhesion as claim 4, wherein, shape of said comparative pattern is circle, ellipse, polygon or the combination thereof.

6. The strap sticker for body adhesion as claim 1, wherein, said glue film is laid over a local surface area on said elastic body in wave form or concentric form; cross sectional shape of opening of said observation well is circle, ellipse, polygon or the combination thereof.

7. The strap sticker for body adhesion as claim 1, wherein, said elastic body may be made of Polyurethane, Polyethylene, Polypropylene, polyvinyl chloride, Ethylene Vinyl Acetate copolymer, Ethylene Vinyl Acetate copolymer, poly vinyl alcohol, Polyamide, Thermoplastic Polyurethane, Poly(ethylene terephthalate) or the arbitrary combination thereof.

8. A strap sticker for body adhesion, including:
an elastic body, having an outer layer and an inner layer with different elastic coefficients, and an observation well penetrating said outer layer and said inner layer; and
a force showing bar, which is connected between opposed walls on two ends of said observation well, and has a structurally weak point for responding extension level of said elastic body.

9. The strap sticker for body adhesion as claim 8, wherein, said structurally weak point indicates a part with smaller thickness, cross sectional area, or strength than other part in the force showing bar.

10. The strap sticker for body adhesion as claim 8, wherein, said elastic body may be made of Polyurethane, Polyethylene, Polypropylene, polyvinyl chloride, Ethylene Vinyl Acetate copolymer, Ethylene Vinyl Acetate copolymer, poly vinyl alcohol, Polyamide, Thermoplastic Polyurethane, Poly(ethylene terephthalate) or the arbitrary combination thereof.

11. A strap sticker for body adhesion, including:
an elastic body, having an outer layer and an inner layer with different elastic coefficients, and an observation well penetrating said outer layer and said inner layer; and
an extension level identification tag, which is extended from said inner layer to be exposed to said observation well, well diameter of said observation well in said inner layer being larger than that in said outer layer to form an indent space inside said observation well, such that said extension level identification tag being indented in said indent space to reduce level of exposure to said observation well as said elastic body does not bear force to extend; said extension level identification tag being extended outside said indent space and exposed to said observation well as said elastic body bearing force to extend.

12. The strap sticker for body adhesion as claim 11, wherein, portion of said extension level identification tag exposed to said observation well is marked with text or pattern.

13. The strap sticker for body adhesion as claim 12, wherein, said elastic body may be made of Polyurethane, Polyethylene, Polypropylene, polyvinyl chloride, Ethylene Vinyl Acetate copolymer, Ethylene Vinyl Acetate copolymer, poly vinyl alcohol, Polyamide, Thermoplastic Polyurethane, Poly(ethylene terephthalate) or the arbitrary combination thereof.
